(19) United States
(12) Patent Application Publication
(10) Pub. No.: US 2011/0142901 A1
(43) Pub. Date: Jun. 16, 2011

(54) SUSTAINED RELEASE COMPOSITION

(76) Inventors: Malcolm BRANDON, Balwyn (AU); Serge R. MARTINOD, Groton, CT (US)

(21) Appl. No.: 12/860,470

(22) Filed: Aug. 20, 2010

Related U.S. Application Data
(63) Continuation of application No. 11/628,036, filed on Aug. 22, 2007, now abandoned.

(30) Foreign Application Priority Data
May 31, 2004 (AU) 2004902893

(51) Int. Cl. A61M 31/00; A61K 9/14; A61K 38/09; A61K 9/00; A61P 5/26; A61P 3/00; A61P 15/08; A61P 15/00; A61P 35/00; A23L 1/31

(52) U.S. Cl. 424/422; 424/489; 514/10.3; 514/10.4; 514/10.6; 424/484; 426/645

(57) ABSTRACT

A sustained release apparatus including at least one sustained release mini-implant or pellet; the or each mini-implant or pellet including: a sustained release support material; and a pharmaceutical composition including a Luteinising Hormone Releasing Hormone (HLRH) agonist and/or antagonist component the size and/or number and/or payload of mini-implant(s) or pellet(s) providing, release of LHRH agonist and/or antagonist at, or above, a desired threshold level for treatment of a selected indication, the apparatus providing approximately zero order release of the LHRH agonist and/or antagonist.

The present invention relates to a sustained release apparatus, and in particular a sustained release apparatus in an implant or pellet form. More specifically, the present invention relates to a sustained release apparatus which provides for treatment of various indications associated with hormone production in animals including humans.

A number of drug delivery systems are known in the prior art.

For example, a controlled drug-release preparation using as a carrier a hydrophobic polymer material, which is non-degradable after administration into the living body. There are two methods of controlling release of a drug from such preparation; one, using an additive such as an albumin (Japanese patent publication (Tokkohei) No. 61959/1995), and another, by forming an outer layer consisting of hydrophobic polymer alone (Japanese patent publication (Tokkohei) No. 187994/1995).

However, where a disease indication requires the achievement of a high threshold blood plasma level and/or requires the delivery of multiple pharmaceuticals and/or requires sustained release to be continued over an extended period at high levels, the drug delivery systems known in the prior art generally exhibit insufficient drug carrying capacity and release rate that are too slow to achieve high blood levels over a sustained time period.

Whilst it is theoretically possible to increase the amount of active delivered by increasing the size of the drug delivery systems in one or more dimensions (e.g. length or diameter), this may not achieve the anticipated result, e.g. as this may lead to “dose dumping” which may be harmful or even lethal to the animal to be treated. Alternatively the large size of the apparatus may prevent its use even with relatively large animals, in particular cattle.

For example, such drug delivery implants may be placed subcutaneously in the ear of an animal. This may be physically impossible where the size of the implant becomes too large.

Vaccination against the hypothalamic hormone luteinising hormone releasing hormone, for example, (referred to herein as “LHRH”, also known as GnRH) has been demonstrated as an immunological method of controlling reproduction since the early 1970's.

De-sexing operations are the most widely practised surgical procedures in veterinary medicine and livestock animal management. A significant proportion of both sexes of domestic livestock and companion animals are routinely surgically de-sexed to prevent a variety of undesirable characteristics which accompany sexual maturity. The traits include fighting, wandering, sexual behaviour, loss of condition, tumours of reproductive organs and pregnancy.

Similarly, the control and treatment of disorders of the gonads and other reproductive organs, of both humans and animals, such as testicular cancer, breast cancer, prostate cancer, ovarian cancer, prostate enlargement or endometriosis is of significance.

For domestic animals, such as dogs, there is a need for antifertility agents that are safe and less invasive than gonadectomy and, in the case of potential breeding stock, completely reversible. To be acceptable, contraceptives for animals must interfere with fertility but have negligible side-effects during use. Reversible contraceptives also need to have negligible side-effects after withdrawal for both treated animals and their offsprings. For males, in particular, very few options are currently available that would satisfy many of these criteria.

Superagonists of gonadotrophin-releasing hormone (GnRH) have long been seen as an option because long-term administration markedly reduces the secretion of luteinising hormone.

Similarly during sexual development and when mature, boars accumulate substances, predominantly androstenone and skatole, in their fatty tissue that are regarded as the main contributors to boar taint in pork. To avoid tainting of the meat, boars destined for fresh meat consumption have until recent years, been slaughtered before sexual maturity. In other countries taint is overcome by castration of the boar before weaning. However, castration results in significant reductions in growth performance and excess deposition of fat. Because boar taint increases with sexual maturity, the increase in slaughter weight has been associated with an increase in the risk of boar taint. One method of inhibiting sexual development and boar taint is immunization against LHRH. However, most vaccine regimens reported to date have been inappropriate because they have required many injections or the site reactions that occurred after injection of adjuvants required a long vaccination-to-slaughter interval. Further the vaccine regimens require prolonged periods of treatment which results in long periods of reduced testosterone levels in the animal. In the case of livestock animals, such as boars, this results in significant reductions in growth performance leading to reduced carcass weights and quality at slaughter.

Moreover such vaccine regimens have failed to deliver adequate antibody response to ensure that no treated animals will develop boar taint.

For example, in one study, vaccination of male pigs has resulted in variable suppression of testis development and suppression of boar taint. An LHRH conjugate used for vaccination gave an antibody response in only 90% of 20 vaccinated pigs.

Modelling has suggested that, in order to be acceptable to most consumers, threshold values of 0.5-1.0 μg/g of fat and 0.20 μg/g of fat for androstenone and skatole respectively need to be sought (Bonneau et al, “An international study on the importance of androstenone and skatole for boar taint: IV. Simulation studies on consumer dissatisfaction with entire male pork and the effect of sorting carcasses on the slaughter line, main conclusions and recommendations”—Meat Sci (2000) 54: 285-295.)

It would accordingly be a significant advance in the prior art if a pharmaceutical composition could be provided which had the effect of successfully treating 100% of animals.

Moreover, in the prior art there is a significant lag time between treatment and response. If it were possible to generate a more rapid onset of activity and a persistence of active response, the nature and scope of potential indications that may be treated would be significantly extended.

It is, accordingly, an object of the present invention to overcome or at least alleviate one or more of the difficulties and deficiencies related to the prior art.

In a first aspect of the present invention, there is provided a sustained release apparatus including at least one sustained release mini-implant or pellet;
the or each mini-implant including;
a sustained release support material; and
a pharmaceutical composition including
   a Luteinising Hormone Releasing Hormone (LHRH) agonist and/or antagonist component;
      the size and/or number and/or payload of mini-implant(s) or pellet(s) providing, release of LHRH agonist and/or antagonist at, or above, a desired threshold level for treatment of a selected indication, the apparatus providing approximately zero order release of the LHRH agonist and/or antagonist.

Applicants have surprisingly found that the sustained release apparatus may generate, in use, a rapid onset of activity as well as a persistence in active response.

Accordingly, the sustained release apparatus is particularly suitable for the control of reproduction and the control and treatment of the gonads and other reproduction organs, of both humans and animals, including prostate cancer, testicular cancer, prostate enlargement, ovarian cancer, endometriosis and the like.

The sustained release apparatus may further be utilised in the regulation of characteristics associated with sexual maturation of a male or female animal, and/or during seasonal breeding cycles, e.g. inhibition of physical and/or behavioural characteristics including, for example, aggression including amounting or fighting, wandering, loss of condition, sexual behaviour, oestrus cycling, fertility and pregnancy.

Through the control of reproduction, particularly over a period of short duration, it is possible to control unwanted organoleptic characteristics including the phenomenon of meat taint, particularly boar taint in pork from male pigs. Moreover, as the treatment is of such a short duration, the method may function to improve the carcass quality of the animal to be treated.

Most testosterone production occurs in the testes, and a considerably smaller amount is produced in the adrenal glands. Applicants have surprisingly found that the sustained release apparatus may result in blood levels of agonist and/or antagonist sufficient to reduce levels of testosterone derived from both the testes and the adrenal glands.

Without wishing to be bound by theory, the applicants believe that reduction of both testicular derived testosterone and adrenal gland derived testosterone results in a concomitant reduction in the level of skatole.

Preferably the sustained release mini-implant(s) or pellet(s) in combination may provide a blood level of pharmaceutical active at least equal to a predetermined threshold for an extended period, e.g. of approximately 1 to 52, preferably 2 to 48 weeks, more preferably 2 to 4 weeks and most preferably 1 to 2 weeks, for a selected active.

Preferably the sustained release apparatus may provide a blood level of agonist and/or antagonist is sufficient to reduce or eliminate boar taint.

Preferably the LHRH agonist and/or antagonist content is approximately 1 to 20 mg, more preferably 2 to 10 mg, most preferably 2 to 6 mg.

Preferably the length of the mini-implant or pellet is approximately 0.05 to 1.5 cm, more preferably 0.1 to 1 cm, most preferably 0.1 to 0.5 cm.

Preferably the internal diameter of the mini-implant or pellet is approximately 0.5 to 2.0 mm, more preferably 0.75 to 1.75 mm, most preferably 0.8 to 1.4 mm.

Preferably the outside diameter of the mini-implant or pellet is approximately 0.5 to 3.0 mm, more preferably 1.0 to 2.0 mm, most preferably 1.0 to 1.6 mm.

Each sustained release mini-implant or pellet according to the present invention is biocompatible and may be biodegradable.

The composition, as described above, includes at least one LHRH agonist and/or antagonist component.

The LHRH agonist and/or antagonist component may be of any suitable type. Active derivatives of LHRH may be used. Derivatives include precursors fragments, parts, portions, chemical equivalents, salts, mutants, homologs and analogs from natural, synthetic or recombinant sources, including fusion proteins and DNA/RNA sequences coding for active. The LHRH agonists [D-Trp6] LHRH, Decapeptyl, Leuprolide, Zolandex, Buserelin or Deslorelin (D-Trp⁶-Pro⁹-des-Gly¹⁰-LHRH ethyl-amide), and salts thereof, have been found to be suitable.

The LHRH antagonists Ganirelix, Abarelix, Cetrorelix acetate (Ac-D-NaI(2) (4CI), D-Pal(3)3, D-Cit6, D-Ala10) LHRH or Cetrorelix pamoate, and salts thereof, have been found to be suitable.

The LHRH agonist/antagonist may be present in relatively large amounts in the pharmaceutical composition. The LHRH agonist/antagonist may be present in amounts of approximately 40 to 70%, preferably approximately 40 to 60% by weight, based on the total weight of the pharmaceutical composition.

The pharmaceutical composition may further include a secondary pharmaceutically active component. The secondary pharmaceutically active component may be exemplified by, but not limited to, one or more selected from the group consisting of:

| | |
|---|---|
| Acetonemia preparations | Anabolic agents |
| Anaesthetics | Analgesics |
| Anti-acid agents | Anti-arthritic agents |
| Antibodies | Anti-convulsivants |
| Anti-fungals | Anti-histamines |
| Anti-infectives | Anti-inflammatories |
| Anti-microbials | Anti-parasitic agents |
| Anti-protozoals | Anti-ulcer agents |
| Antiviral pharmaceuticals | Behaviour modification drugs |
| Biologicals | Blood and blood substitutes |
| Bronchodilators and | Cancer therapy and related |
| expectorants | pharmaceuticals |
| Cardiovascular pharmaceuticals | Central nervous system pharmaceuticals |
| Coccidiostats and coccidiocidals | Contraceptives |
| Contrast agents | Diabetes therapies |
| Diuretics | Fertility pharmaceuticals |
| Growth hormones | Growth promoters |
| Hematinics | Hemostatics |
| Hormone replacement therapies | Hormones and analogs |
| Immunostimulants | Minerals |
| Muscle relaxants | Natural products |
| Nutraceuticals and nutritionals | Obesity therapeutics |
| Ophthalmic pharmaceuticals | Osteoporosis drugs |
| Pain therapeutics | Peptides and polypeptides |
| Respiratory pharmaceuticals | Sedatives and tranquilizers |
| Transplantation products | Urinary acidifiers |
| Vaccines and adjuvants | Vitamins |

The secondary pharmaceutically active component may include a water-insoluble pharmaceutical, a water-soluble pharmaceutical or mixtures thereof.

The sustained release apparatus, in use in entire male animals, may provide a blood level of LHRH agonist and/or antagonist sufficient to significantly reduce skatole levels. Preferably, the skatole level is reduced to below 0.2 μg/g of fat.

The term “significantly reduced” as used herein refers to a reduction sufficient to reduce or eliminate boar taint.

The sustained release apparatus, in use in entire male animals, may provide a blood level of LHRH agonist and/or antagonist sufficient to concomitantly significantly reduce testosterone levels. Preferably, the testosterone level is reduced to below approximately 1 ng/mL of serum. Most preferably, the testosterone level is reduced to below 0.2 ng/mL of serum.

The sustained release apparatus, in use in entire male animals, may provide a blood level of LHRH agonist and/or antagonist sufficient to concomitantly significantly reduce androstenone levels. Preferably, the androstenone level is reduced to below approximately 0.5 μg/g of fat. Most preferably, the androstenone level is reduced to below 0.2 μg/g of fat.

The water-soluble pharmaceutical actives useful in the sustained release apparatus according to the present invention include such drugs as peptides, polypeptides, proteins, glycoproteins, polysaccharides, hormones and nucleic acids.

A combination of an LHRH agonist/antagonist component and a growth hormone component is particularly preferred.

A synthetic growth hormone, e.g. Recombinant Porcine Somatotropin (rPST) may be used.

The secondary pharmaceutically active component may be present in the pharmaceutical composition in any suitable amounts. The secondary pharmaceutically active component may be present in amounts from approximately 8 to 50% by weight, preferably approximately 15 to 30% by weight, based on the total weight of the pharmaceutical composition.

In a preferred form, the sustained release apparatus may include two or more mini-implants of similar or different sizes and of similar or different formulation.

The mini-implants may be administered simultaneously in a single treatment. The mini-implants may be administered via a single treatment, e.g. a single injection or the like as discussed below.

The pharmaceutical composition according to the present invention, may further include a carrier for the LHRH agonist/antagonist component.

The pharmaceutical carrier may be selected to permit release of the pharmaceutically active component over a shortened or extended period of time from the composition.

The carrier may include a water-soluble or water-insoluble substance.

A water-soluble substance is a substance which plays a role of controlling infiltration of water into the inside of the drug dispersion. There is no restriction in terms of the water-soluble substance so long as it is in a solid state (as a form of a preparation) at the body temperature of an animal or human being to which it is to be administered, and a physiologically acceptable, water-soluble substance.

One water-soluble substance, or a combination of two or more water-soluble substances may be used. The water-soluble substance specifically may be selected from one or more of the group consisting of synthetic polymers (eg. polyethylene glycol, polyethylene polypropylene glycol), sugars (eg. sucrose, lactosemannitol, glucose, sodium chondroitin sulfate), polysaccharides (e.g. dextran), amino acids (eg. glycine and alanine), mineral salts (eg. sodium chloride), organic salts (eg. sodium citrate) and proteins (eg. gelatin and collagen and mixtures thereof).

In addition, the water-soluble substance may include a substance which is water-soluble and has any activity in vivo such as low molecular weight drugs, peptides, proteins, glycoproteins, polysaccharides, or an antigenic substance used as vaccines, i.e. water-soluble drugs.

In addition, when the water-soluble substance is an amphipathic substance, which dissolves in both an organic solvent and water, it has an effect of controlling the release of, for example, a lipophilic drug by altering the solubility thereof. An amphipathic substance includes, but not limited to, one or more selected from the group consisting of polyethylene glycol or a derivative thereof, polyoxyethylene polyoxypropylene glycol or a derivative thereof, fatty acid ester and sodium alkylsulfate of sugars, and more specifically, polyethylene glycol, polyoxy stearate 40, polyoxyethylene[196]polyoxypropylene [67]glycol, polyoxyethylene[105] polyoxypropylene[5]glycol, polyoxyethylene[160] polyoxypropylene[30]glycol, sucrose esters of fatty acids, sodium lauryl sulfate, sodium oleate, sodium chloride, sodium desoxycholic acid (or sodium deoxycholic acid (DCA)) of which mean molecular weights are more than 1500.

Polyoxyethylene polyoxypropyleneglycol, sucrose, lactose, mannitol, dextran, sodium chloride or DCA or a mixture of two or more thereof are preferred.

The water-insoluble carrier may be selected from one or more of the group of water insoluble polymers, resins and latexes including water-insoluble acrylates, methacrylates and other carboxy polymers, waxes, lipids including phospholipids and lipoproteins.

The pharmaceutical carrier may constitute from approximately 1% to 30% by weight, preferably approximately 1% to 15% by weight, based on the total weight of the pharmaceutically active composition.

Each sustained release mini-implant or pellet may include additional carrier or excipients, lubricants, fillers, plasticisers, binding agent, colourants and stabilising agents.

Suitable fillers may be selected from the group consisting of talc, titanium dioxide, starch, kaolin, cellulose (microcrystalline or powdered) and mixtures thereof.

Each sustained release mini-implant or pellet according to the present invention may be of the covered rod or matrix type. A covered rod-like shape is preferred.

The size, number and payload of the mini-implant(s) or pellet(s) may vary with the pharmaceutically active component and/or antigen/adjuvant selected.

The optimum combination of size number and payload may be established through simple experiment.

For example, each sustained release mini-pellet may be approximately 0.1 to 0.5 times, preferably approximately 0.20 to 0.40 times, the length of a single rod shaped implant, and capable of providing the desired threshold blood level, depending on the pharmaceutical active selected.

For example, in veterinary applications, a typical pig implant according to the present invention may have dimensions of approximately 1.5 mm outer diameter×1 cm in length, administered as 4 implants of 0.25 cm in length.

The sustained release delivery apparatus may take the form of a covered rod or dispersed matrix structure. Such a multi mini-pellet system permits the treatment of diseases over an extended period with pharmaceutically active components which have heretofore not been applicable to such diseases as it has not been possible to achieve the required threshold blood plasma levels to be efficacious and to maintain those blood levels over a sufficient period of time.

Preferably the sustained release delivery apparatus may provide approximately zero order release of LHRH agonist/antagonist.

The sustained release support material may take the form of a support matrix or rod, preferably a covered rod structure. The sustained release support material may take the form of an open ended cylindrical rod.

The sustained release support material may be formed from a biodegradable or biocompatible material, preferably a biocompatible hydrophobic material. The biocompatible material may be selected from the group consisting of polyesters, polyamino acids, silicones, ethylene-vinyl acetate copolymers and polyvinyl alcohols. Preferably the sustained release support material is a silicone material. A silicone rod is preferred. The silicone material may be a porous silicon or Biosilicon material, for example as described in International patent application PCT/GB99/01185, the entire disclosure of which is incorporated herein by reference. A mesoporous, microporous or polycrystalline silicon or mixtures thereof may be used.

Biodegradable polymers that may be employed in the present invention may be exemplified by, but not limited to, polyesters such as poly(lactic acid-glycolic acid) copolymers (PLGA), hydrophobic polyamino acids such as polyaranin, polyleucine, polyanhydride, poly(glycerol-sebacate)(PGS), Biopol, Pluronic polyols (Poloxamers) and the like. The hydrophobic polyamino acids mean polymers prepared from hydrophobic amino acids.

Nonbiodegradable polymers that may be employed in the present invention may be exemplified by, but not limited to, silicones, polytetrafluoroethylenes, polyethylenes, polypropylenes, polyurethanes, polyacrylates, polymethacrylates such as polymethylmethacrylates, etc., ethylene-vinyl acetate copolymers, and others.

More preferably a silicone elastomer as described in International patent application PCT/AU02/00865, to applicants (the entire disclosure of which is incorporated herein by reference), may be used. For example the silicone elastomer may be formed from a methyl-vinyl siloxane polymer including a fumed silica as reinforcing filler. The sustained release support material may comprise approximately 40 to 65% by weight, preferably approximately 45 to 60% by weight, of the sustained release apparatus, the remainder formed of the pharmaceutical composition.

Suitable binding agents include polyvinyl pyrrolidine, hydroxypropyl cellulose and hydroxypropyl methyl cellulose and mixtures thereof.

The sustained release implant according to the present invention may have a rod-like shape, for example it is selected from circular cylinders, prisms, and elliptical cylinders. When the device is administered using an injector-type instrument, a circular cylindrical device is preferred since the injector body and the injection needle typically have a circular cylindrical shape.

The sustained release implant according to the present invention may be manufactured according to copending International patent application PCT/AU2002/000868 entitled “Preparation of sustained release pharmaceutical composition”, to Applicants, the entire disclosure of which is incorporated herein by reference.

The inner layer of the pharmaceutical formulation of the present invention, viewed in right section, may contain two or more layers containing different water-soluble pharmaceuticals. These layers may take the form of concentric circles with a single center of gravity or may appear as a plural number of inner layers whose respective centers of gravity lie at different points in the cross section. When the pharmaceutical formulation contains more than one inner layer there may be one or more pharmaceuticals present in the inner layers. For example, the pharmaceuticals may be present such that each layer contains a different pharmaceutical or there is more than one pharmaceutical in one or all of the inner layers.

The size of the pharmaceutical formulation of the present invention may, e.g. in the case of subcutaneous administration, be relatively small, e.g. ¼ to 1/10 normal size. For example using an injector-type instrument, the configuration may be circular cylindrical, and the cross-sectional diameter in the case is preferably 0.2 to 16 mm, the axial length being preferably approximately 0.2 to 7.5 mm, preferably approximately 0.5 to 5 mm, more preferably approximately 1 to 4 mm.

Sustained release implants according to the present invention may preferably have a double-layer structure, in order to achieve long-term zero-order release. The double layer structure may include
a LHRH agonist and/or antagonist containing inner layer; and
a water impermeable outer layer.

The water impermeable outer layer may be formed of a silicone material. More preferably water-impermeable outer layers may be formed from a liquid coating composition including a liquid siloxane component.

Applicants have surprisingly found that the sustained release mini-implants having a double layer structure exhibit an unexpected release profile. Contrary to expectations, the maximum serum levels vary with the length of implant, not merely the time period over which sustained release is maintained (see Tables 4A and 4B). Whilst we do not wish to be restricted by theory, it is postulated that, particularly for small molecules, release is occurring not only from the open ends of the covered rod implant but also through the water-impermeable outer layer.

Desirably the rod-like implant includes an outer coating layer. The thickness of the outer layer should be selected as a function of the material properties and the desired release rate. The outer layer thickness is not critical as long as the specified functions of the outer layer are fulfilled. The outer layer thickness is preferably approximately 0.05 mm to 3 mm, more preferably 0.05 mm to 0.25 mm, and most preferably 0.05 mm to 0.1 mm.

A pharmaceutical formulation with an open end at one terminal only may be fabricated by dipping one terminal of the pharmaceutical formulation into a solution which dissolves the outer-layer material and drying it, or by covering one terminal end of the pharmaceutical formulation with a cap made from the outer-layer material. In addition, the fabrication may comprise insertion of the inner layer into an outer-layer casing with a closed-end at one terminal, which are separately produced, and also formation of the inner layer in said casing.

In a preferred embodiment of this aspect of the present invention, the sustained release apparatus may further include
a marker component.

Applicants have found that to ensure the removal of the sustained release apparatus prior to, or at, slaughter, a marker component is desirable.

The marker component may include a dye component or metal marker component. The marker component may include a radio-opaque component, e.g. of the type described in International patent application no. PCT/AU02/01661 to Applicants, the entire disclosure of which is incorporated herein by reference.

In a further aspect of the present invention, there is provided a sustained release apparatus including at least one sustained release mini-implant or pellet;
the or each mini-implant or pellet including;
a sustained release support material; and
a pharmaceutical composition including
   a Luteinising Hormone Releasing Hormone (LHRH) agonist and/or antagonist component
      the size and/or number and/or payload of mini-implant(s) or pellet(s) providing, release of LHRH agonist and/or antagonist at, or above, a desired threshold level for regulation of characteristics associated with the onset of sexual maturation of an animal and/or seasonal breeding activity, the apparatus providing a blood level of LHRH agonist and/or antagonist sufficient to significantly reduce skatole levels.

Preferably the blood level of LHRH agonist and/or antagonist is sufficient to concomitantly significantly reduce skatole and testosterone levels. More preferably the blood level of LHRH agonist and/or antagonist is sufficient to comcomitantly significantly reduce skatole, testosterone and androstenone levels.

Preferably the LHRH agonist and/or antagonist content is approximately 1 to 20 mg, more preferably approximately 2 to 6 mg.

In a still further preferred embodiment, there is provided a sustained release kit including
a plurality of sustained release mini-implants or pellets packaged for delivery in a single treatment;
each mini-implant or pellet including
a sustained release support material; and
a pharmaceutical composition including
   an LHRH agonist and/or antagonist component;
      the size and/or number and/or payload of mini-implants or pellets providing release of LHRH agonist and/or antagonist at, or above, a desired threshold level for treatment of a selected indication, the plurality of sustained release mini-implants or pellets providing approximately zero order release of the LHRH agonist and/or antagonist.

Preferably, the number of mini-implants or pellets is 3 or more, more preferably 4 or more.

In a further preferred embodiment, each mini-implant or pellet includes
a LHRH agonist/antagonist containing inner layer; and
a water impermeable outer layer.

Optionally the sustained release kit further includes a sustained release delivery apparatus. For example, in veterinary applications, an injector instrument for subcutaneous delivery of standard size pellets may be used as the sustained release delivery apparatus.

The multiple mini-pellets may be delivered utilising a standard injector instrument, preferably in a single cartridge for use in a standard injector instrument which in turn disperse as individual mini-pellets within the body of the animal to be treated.

In a further preferred form of the present invention, the plurality of sustained release mini-implants or pellets may be provided in a biodegradable sheath. The biodegradable sheath may be formed of a water-soluble material.

The water-soluble material utilised in the biodegradable sheath may be selected from one or more of the water-soluble substances described below.

In a further aspect of the present invention there is provided a method for the therapeutic or prophylactic treatment of an indication in an animal (including a human) requiring such treatment, which method includes administering to the animal a sustained release apparatus including one or more sustained release mini-implant(s) or pellet(s);
the or each mini-implant or pellet including
a sustained release support material; and
a pharmaceutical composition including
   an LHRH agonist and/or antagonist component;
      the size and/or number and/or payload of mini-implants or pellets providing release of LHRH agonist and/or antagonist at, or above, a desired threshold level for treatment of a selected indication, the apparatus providing approximately zero order release of the LHRH agonist and/or antagonist.

Preferably, the sustained release apparatus includes a plurality of mini-implants or pellets which in combination provide a blood level of LHRH agonist and/or antagonist at least equal to a predetermined threshold for an extended period.

Preferably, the treatment provides a reduction in hormone production and/or a reduction in the production of hormone-associated compounds.

As stated above, it has been found that the sustained release apparatus may provide, in use, a rapid onset of activity and a persistence in effect. This may be achieved by administration of the sustained release apparatus in a one-shot treatment and without the necessity for follow-up treatments.

The method of administration may include oral, subcutaneous or intramuscular injection, intradermal injection, intraperitoneal injection, intraocular or in the ear, intranasal insertion or indwelling, intravaginal or intradwelling, intrarectal insertion or indwelling, for example as a suppository or utilising oral administration.

The animals to be treated may be selected from the group consisting of sheep, cattle, goats, horses, camels, pigs, dogs, cats, ferrets, rabbits, marsupials, buffalos, yacks, primates, humans, birds including chickens, ducks, geese and turkeys, rodents including rats and mice, fish, reptiles and the like.

The method according to the present invention is particularly applicable to larger animals, e.g. cattle, sheep, pigs, dogs and humans where high dosage levels are required to achieve the prerequisite threshold pharmaceutical active blood levels for successful treatment of selected indications.

Further as stated above, the sustained release apparatus is particularly suitable for the regulation of reproduction and the control and treatment of the gonads and other reproduction organs, of both humans and animals, including treatment of disease indications including prostate cancer, testicular cancer, prostate enlargement, ovarian cancer, endometriosis and the like.

The sustained release apparatus is further suitable for the regulation of characteristics associated with the onset of sexual maturation of an animal and/or seasonal breeding activity.

Through the control of reproduction, particularly over a period of short duration, it is possible to control unwanted organoleptic characteristics including the phenomenon of meat taint, particularly boar taint in pork from male pigs. Moreover, as the treatment is of such a short duration, the method may function to improve the carcass quality of the animal to be treated.

Accordingly, in a preferred embodiment of this aspect of the present invention there is provided a method for regulating sexual reproduction in animals, including humans, which method includes administering to the animal to be treated, a sustained release apparatus including at least one sustained release mini-implant or pellet;
the or each mini-implant including;
a sustained release support material; and
a pharmaceutical composition including
   an LHRH agonist and/or antagonist component;
      the size and/or number and/or payload of mini-implants providing release of LHRH agonist and/or antagonist at, or above, a desired threshold level for treatment of a selected indication, the apparatus providing approximately zero order release of the LHRH agonist and/or antagonist.

The method may be utilised to temporarily or permanently control the reproductive capacity of an animal, particularly a male animal. Where temporary treatment is required, cessation of treatment will generally provide a return to normal sexual activity by the treated animal.

For temporary applications, the mini-implants, when required, may be easily removed (e.g. surgically), as they do not migrate in the body and do not result in a tissue depot of pharmaceutical active.

The method may further be utilised to regulate physical and/or behavioural patterns in male and female animals associated with the onset of sexual maturity, or during seasonal breeding cycles.

In a further preferred embodiment, there is provided a method of inhibiting the growth of cells which are regulated, directly or indirectly by LHRH, which method includes administering to the animal to be treated, a sustained release apparatus including at least one sustained release mini-implant or pellet;
the or each mini-implant or pellet including;
a sustained release support material; and
a pharmaceutical composition including
   an LHRH agonist and/or antagonist component;
      the size and/or number and/or payload of mini-implants providing release of LHRH agonist and/or antagonist at, or above, a desired threshold level for treatment of a selected indication, the apparatus providing approximately zero order release of the LHRH agonist and/or antagonist.

The cells to be treated may be selected from testicular cells, breast cells, prostate cells, ovarian cells or oncofoetal cells, particularly malignant forms of such cells.

The cells to be treated may be hyperplastic cells including prostate or endometrial cells.

The cells to be treated may be of animal, including human, origin.

The sustained release apparatus according to the present invention may be preferably utilised to improve carcass quality in livestock, including in particular the elimination of meat taint, particularly boar taint, in pork.

Accordingly, in a still further embodiment of this aspect of the present invention there is provided a method for improving carcass quality in animals which method includes
administering to an entire animal to be treated at a prescribed time and for a preselected short duration, a sustained release apparatus including at least one sustained release mini-implant or pellet;
the or each mini-implant or pellet including;
a sustained release support material; and
a pharmaceutical composition including
   an LHRH agonist and/or antagonist component;
      the size and/or number and/or payload of mini-implants or pellets providing release of LHRH agonist and/or antagonist at, or above, a desired threshold level for treatment of a selected indication, the apparatus providing approximately zero order release of the LHRH agonist and/or antagonist.

Applicants have surprisingly found that, given the accelerated onset of LHRH agonist activity and the extended duration of activity, treatment with a sustained release apparatus may be reduced to one of a short duration, e.g. approximately 1 to 2 weeks. Moreover initiation of treatment may be delayed until, for example, approximately 2 to 4 weeks prior to slaughter. Applicants have further established that administration may be conducted on a single treatment basis without the necessity of further treatment and will cause substantially 100% of the animals to exhibit the desired effect.

At present, where castration is used for livestock management, breeding animals are selected at an immature age and may subsequently kept separate from the rest of the herd, which increases farming costs. The present invention may allow the selection of breeding animals to be delayed until the animal has reached sexual maturity, eliminating the need for the breeding animals to be segregated. In addition, the physical characteristics of the mature animal may be taken into account in selecting the breeding animal. Indeed, a selection may be made even after treatment as the effects of the apparatus are reversible.

In a still further embodiment of this aspect of the present invention there is provided a method of suppressing or eliminating boar taint which method includes
administering to an entire male pig at a prescribed time and for a preselected short duration, a sustained release apparatus including at least one sustained release mini-implant or pellet;
the or each mini-implant or pellet including;
a sustained release support material; and
a pharmaceutical composition including
   a LHRH agonist and/or antagonist component
      the size and/or number and/or payload of mini-implants or pellets providing release of LHRH agonist and/or antagonist sufficient to produce a blood level thereof which significantly reduces skatole levels.

Preferably the apparatus provides zero order release of the LHRH agonist and/or antagonist.

The method may provide a blood level of LHRH agonist and/or antagonist sufficient to significantly reduce skatole levels. Preferably, the skatole level is reduced to below approximately 0.2 μg/g of fat.

The method may provide a blood level of LHRH agonist and/or antagonist sufficient to concomitantly significantly reduce testosterone levels. Preferably, the testosterone level is reduced to below approximately 1 ng/mL of serum. Most preferably, the testosterone level is reduced to below 0.2 ng/mL of serum.

The method may provide a blood level of LHRH agonist and/or antagonist sufficient to concomitantly significantly reduce androstenone levels. Preferably, the androstenone level is reduced to below approximately 0.5 μg/g of fat. Most preferably, the androstenone level is reduced to below 0.2 μg/g of fat.

In another embodiment of this aspect of the present invention there is provided a method of regulating characteristics associated with the onset of sexual maturation of an animal and/or seasonal breeding activity, which method includes administering to the animal to be treated, a sustained release apparatus including at least one sustained release mini-implant or pellet;
the or each mini-implant or pellet including;
a sustained release support material; and
a pharmaceutical composition including
   an LHRH agonist and/or antagonist component;
      the size and/or number and/or payload of mini-implant(s) or pellet(s) providing, release of LHRH agonist and/or sufficient to provide a blood level thereof which significantly reduce skatole levels.

Preferably the blood level of LHRH agonist and/or antagonist is sufficient to concomitantly significantly reduce skatole and testosterone levels. More preferably the blood level of LHRH agonist and/or antagonist is sufficient to comcomitantly significantly reduce skatole, testosterone and androstenone levels.

In a further aspect of the present invention there is provided a pig carcass, or part thereof, substantially free of boar taint, produced by the method described above.

It will be understood that by reducing the period of treatment, the entire animal will generally grow considerably faster and more efficiently (with higher muscle production and less fat) than a castrated animal and with greater feed efficiency.

As stated above, the phenomenon of boar taint in part is associated with the accumulation of substances including androstenone and skatole in the fatty tissue of male pigs. The substantial elimination of boar taint requires testosterone levels to be reduced generally to less than approximately 2 ng/mL of serum and the pheromones androstenone and skatole to less than 0.5-1.0 μg/g and 0.2 μg/g of fat tissue respectively (reference J Animal Science, 2001 79: 2524-2535). Applicants have surprisingly found that treatment with the sustained release apparatus according to the present invention permits the rapid onset of effect sufficient to reach and maintain the desired reduced levels at significantly lower levels than previously reported in the published literature.

It will be understood that the invention disclosed and defined in this specification extends to all alternative combinations of two or more of the individual features mentioned or evident from the text or drawings. All of these different combinations constitute various alternative aspects of the invention.

The present invention will now be more fully described with reference to the accompanying examples. It should be understood, however, that the description following is illustrative only and should not be taken in anyway as a restriction on the generality of the invention described above.

### EXAMPLE 1

### Studies on the Release of Deslorelin Acetate from Solid Slow Release Implants in Pigs

### Study Number: DSL-P-03-001

### Study Location: Pig Research and Training Centre (PRTC)

Victorian Institute of Animal Science
Department of Primary Industries
600 Sneydes Road
Werribee Victoria Australia 3030

### Implants:

Three different implant formulations were evaluated.

Formulation number 1 consisted of deslorelin acetate with DOC and mannitol as additives.
Formulation number 2 consisted of deslorelin acetate with NaCl and dextran as additives.
Formulation number 3 consisted of deslorelin acetate with no additives.

### Size of Implants and Duration of Treatment:

Groups of pigs were treated with different implant lengths and for different durations.

Pigs were treated with implants measuring 1 cm, 0.5 cm or 0.25 cm in length. All the implants had a diameter of 1.5 mm. Pigs were treated at 14, 18 or 20 weeks of age. All pigs were sacrificed at 22 weeks of age. Thus pigs were treated for either 8, 4 or 2 weeks.

### Measurements:

The primary measure was the level of boar taint substances—androstenone and skatole in body fat. Peripheral blood samples were collected at various times after treatment for the measurement of serum testosterone. Daily feed intake, weekly weight gain and testicle size at slaughter was also measured.

The levels of androstenone and skatole in body fat of pigs were measured by VIAS Endocrinology HPLC Service VIAS SOP Number 840 (which has a minimum detection level for androstenone of 0.2 μg/g of fat, and for skatole of 0.3 μg/g of fat).

Serum testosterone measurements were performed by Fiona Armour, University of Melbourne Veterinary Clinic and Hospital using the Coat-A-Count® Total Testosterone radioimmunoassay supplied by Diagnostic Products Corporation, Los Angeles, Calif., United States of America (which has a minimum detection level of 0.2 ng/mL of serum).

Treatment Groups

| | | Age at Implantation | | | | |
|---|---|---|---|---|---|---|
| | | 14^{1,4,5,6} | | 18^{2,4,5,6} | | 20^{3,4,5,6} |
| | | Weeks | | Weeks | | Weeks |
| | Treatment | Implant Length | | | | |
| Treatments | No. | 1 cm | 1 cm | 0.5 cm | 0.5 cm | 0.25 cm |
| Deslorelin | 1 | 3 | 3 | 3 | 3 | 3 |
| acetate | | | | | | |
| DOC | | | | | | |
| Mannitol | | | | | | |
| Deslorelin | 2 | 3 | 3 | 3 | 3 | 3 |
| acetate | | | | | | |
| NaCl Dextran | | | | | | |
| Deslorelin | 3 | 3 | 3 | 3 | 3 | 3 |
| acetate | | | | | | |
| Control7 | | 3 | | | | |

Notes:
¹Pigs' blood sampled at 14, 18, 20 and 22 weeks of age.
²Pigs' blood sampled at 18, 20 and 22 weeks of age.
³Pigs' blood sampled at 20 and 22 weeks of age.
⁴Boar taint substances measured at 22 weeks of age.
⁵Testicle size measured at 22 weeks of age.
⁶Daily feed intake and weekly weight gain to be measured.
7Control pigs - needle inserted into ear only, no implants used.

### Animals and Groups:

Animals were sourced from PRTC and consisted of male large white/landrace pigs. Male pigs were selected from farrowing weeks 23, 24 and 25 and were 15, 14 and 13 weeks of age respectively at selection.

Pigs were divided into 4 groups based on the timing of treatment.

### Group 1—Treated at 14 Weeks.

12 pigs selected from farrowing week 25—13 weeks of age at selection.
Pigs were implanted at 14 weeks of age.
Pig numbers assigned—31-42.

### Group 2—Treated at 18 Weeks

9 pigs were selected from farrowing week 24—14 weeks of age at selection.
Pigs were treated at 18 weeks of age.
Pig numbers assigned—10-18.

### Group 3—Treated at 20 Weeks

18 pigs were selected from farrowing week 24—14 weeks of age at selection.
Pigs were treated at 20 weeks of age
Pig numbers assigned—19-30, 43-48.

### Group 4—Treated at 18 Weeks

9 pigs were selected from farrowing week 23—15 weeks of age at selection.
Pigs were treated at 18 weeks of age.
Pig numbers assigned—1-9.

Pigs were housed in individual pens in the Experimental Grower Facility—PRTC. Identification—pigs were identified by the ear notches (placed at farrowing) and by ear tags.

### Results

The influence of deslorelin acetate on the elimination of boar taint substances in the fat of pigs and on testosterone levels in the serum of pigs are illustrated in Tables 1A to 1D and Tables 2A to 2F.

TABLE 1A

| | | Androstenone | |
|---|---|---|---|
| Pig No. | Treatment | μg/gram fat | Skatole μg/gram fat |
| 40 | Control | 0.84 | 0.604 |
| 41 | Control | 4.1 | 0.194 |
| 42 | Control | 2.1 | 1 |
| Mean | | 2.35 | 0.60 |

TABLE 1B
1.0 cm Implants

| | 14 weeks | | | | 18 weeks | | |
|---|---|---|---|---|---|---|---|
| Pig No. | Treatment | Androstenone | Skatole | Pig No. | Treatment | Androstenone | Skatole |
| 31 | 1 | 5.0 | 0.697 | 1 | 1 | 4.95 | 0.379 |
| 32 | 1 | 1.1 | 0.145 | 2 | 1 | 5.0 | 0.22 |
| 33 | 1 | 1.7 | 0.405 | 3 | 1 | 5.0 | 0.186 |
| Mean | | 2.60 | 0.42 | | | 4.98 | 0.26 |
| 34 | 2 | 2.4 | 0.10 | 4 | 2 | 5.0 | 0.3825 |
| 35 | 2 | 2.6 | 0.06 | 5 | 2 | 5.0 | 0.07 |
| 36 | 2 | 2.5 | 0.087 | 6 | 2 | 3.2 | 1 |
| Mean | | 2.50 | 0.08 | | | 4.40 | 0.48 |
| 37 | 3 | 0.8 | 0.069 | 7 | 3 | 5.0 | 0.459 |
| 38 | 3 | 1.4 | 0.043 | 8 | 3 | 5.0 | 0.144 |
| 39 | 3 | 2.0 | 0.216 | 9 | 3 | 5.0 | 0.278 |
| Mean | | 1.40 | 0.11 | | | 5.0 | 0.29 |

TABLE 1C
0.50 cm

| | 18 weeks | | | | 20 weeks | | |
|---|---|---|---|---|---|---|---|
| Pig No. | Treatment | Androstenone | Skatole | Pig No. | Treatment | Androstenone | Skatole |
| 16 | 1 | 1.9 | 1.0 | 20 | 1 | 0.84 | 0.063 |
| 17 | 1 | 1.3 | 0.12 | 24 | 1 | 5.0 | 0.452 |
| 18 | 1 | 3.7 | 1.0 | 26 | 1 | 1.8 | 0.852 |
| Mean | | 2.3 | 0.71 | Means | | 2.55 | 0.46 |
| 13 | 2 | 2.1 | 0.136 | 21 | 2 | 1.4 | 0.143 |
| 14 | 2 | 1.7 | 0.499 | 46 | 2 | 1.2 | 0.12 |
| 15 | 2 | 3.0 | 0.288 | 47 | 2 | 4.1 | 0.098 |
| Mean | | 2.37 | 0.31 | Mean | | 2.23 | 0.12 |
| 10 | 3 | 5.0 | 0.241 | 29 | 3 | 1.2 | 0.417 |
| 11 | 3 | 5.0 | 0.180 | 44 | 3 | 2.5 | 0.846 |
| 12 | 3 | 0.37 | 0.074 | 45 | 3 | 0.95 | 0.061 |
| Mean | | 3.46 | 0.17 | Mean | | 1.55 | 0.441 |

TABLE 1D
0.25 cm Implants 20 weeks

| Pig No. | Treatment | Androstenone | Skatole |
|---|---|---|---|
| 25 | 1 | 0.63 | 0.049 |
| 28 | 1 | 2.4 | 0.743 |
| 43 | 1 | 0.68 | 0.06 |
| Mean | | 1.24 | 0.28 |
| 22 | 2 | 3.2 | 0.212 |
| 23 | 2 | 2.4 | 0.213 |
| 30 | 2 | 1.3 | 0.35 |
| Mean | | 2.30 | 0.26 |
| 19 | 3 | 1.1 | 0.125 |
| 27 | 3 | 1.2 | 0.188 |
| 48 | 3 | 1.6 | 0.252 |
| Mean | | 1.30 | 0.19 |

TABLE 2A
The effects of silicone implants containing deslorelin
acetate on serum testosterone in pigs

| | | Serum Testosterone - ng/ml | | | | | | |
|---|---|---|---|---|---|---|---|---|
| | | Age - Weeks | | | | | | |
| Pig No. | Treat | 14 | 15 | 16 | 17 | 18 | 20 | 22 |
| 40 | Control | 0.4 | 1.39 | 4.45 | 2.39 | 0.85 | 1.13 | 4.53 |
| 41 | Control | 3.9 | 2.05 | 1.42 | 1.63 | 1.39 | 3.77 | 2.98 |
| 42 | Control | 2.35 | 1.38 | 2.08 | 2.31 | 3.03 | 2.44 | 5.41 |
| Mean | | 2.22 | 1.61 | 2.65 | 2.11 | 1.76 | 2.45 | 4.31 |

TABLE 2B

| | | Serum Testosterone - ng/ml | | | | | | |
|---|---|---|---|---|---|---|---|---|
| 1.0 cm Implants | | Treated at 14 weeks of age | | | | | | |
| 31 | 1 | 1.76 | 1.48 | 1.72 | 2.01 | 1.47 | 3.29 | 5.26 |
| 32 | 1 | 1.42 | 1.05 | 1.83 | 1.01 | 1.3 | 3.3 | 2.35 |
| 33 | 1 | 2.21 | 1.25 | 1.16 | 1.41 | 0.78 | 2.72 | 2.53 |
| Mean | | 1.80 | 1.26 | 1.57 | 1.48 | 1.18 | 3.10 | 3.38 |
| 34 | 2 | 1.26 | 0.82 | 2.08 | 2.58 | 1.71 | 6.03 | 4.92 |
| 35 | 2 | 3.37 | 1.63 | 4.96 | 2.73 | 1.63 | 5.65 | 6.94 |
| 36 | 2 | 2.28 | 1.62 | 2.84 | 1.79 | 2.9 | 4.52 | 6.57 |
| Mean | | 2.30 | 1.36 | 3.29 | 2.37 | 2.08 | 5.4 | 6.14 |
| 37 | 3 | 1.59 | 1.03 | 1.8 | 1.71 | 1.03 | 1.22 | 2.08 |
| 38 | 3 | 1.76 | 0.68 | 2.76 | 1.53 | 0.68 | 2.41 | 2.28 |
| 39 | 3 | 1.79 | 0.87 | 2.14 | 1.5 | 0.87 | 2.42 | 2.47 |
| Mean | | 1.71 | 0.86 | 2.23 | 1.58 | 0.86 | 2.02 | 2.28 |

TABLE 2C

| | |
|---|---|
| | Serum Testosterone - ng/ml |
| 1.0 cm Implants | Treated at 18 weeks of age |

| | | | | |
|---|---|---|---|---|
| 1 | 1 | 2.72 | 1.72 | 2.87 |
| 2 | 1 | 3.37 | 2.49 | 4.51 |
| 3 | 1 | 1.8 | 2.07 | 1.78 |
| Mean | | 2.63 | 2.09 | 3.05 |
| 4 | 2 | 5.02 | 2.57 | 5.65 |
| 5 | 2 | 3.22 | 1.2 | 3.37 |
| 6 | 2 | 3.21 | 1.58 | 2.86 |
| Mean | | 3.82 | 1.78 | 3.96 |
| 7 | 3 | 5.52 | 2.32 | 6.24 |
| 8 | 3 | 1.09 | 1.76 | 2.18 |
| 9 | 3 | 4.47 | 2.35 | 4.85 |
| Mean | | 3.69 | 2.14 | 4.42 |

TABLE 2D

| | |
|---|---|
| | Serum Testosterone - ng/ml |
| 0.5 cm Implants | Treated at 18 weeks of age |

| | | | | |
|---|---|---|---|---|
| 10 | 1 | 3.27 | 3.79 | 3.54 |
| 11 | 1 | 6.21 | 4.41 | 3.79 |
| 12 | 1 | 3.72 | 1.67 | 1.03 |
| Mean | | 4.4 | 3.29 | 2.79 |
| 13 | 2 | 3 | 3.29 | 2.56 |
| 14 | 2 | 2.52 | 5.77 | 1.96 |
| 15 | 2 | 2.22 | 1.25 | 2.62 |
| Mean | | 2.58 | 3.16 | 2.38 |
| 16 | 3 | 4.93 | 3.93 | 2.72 |
| 17 | 3 | 6.62 | 4.82 | 3.69 |
| 18 | 3 | 4.06 | 5.28 | 4.37 |
| Mean | | 5.20 | 4.68 | 3.59 |

TABLE 2E

| Serum Testosterone - ng/ml | | | | | | |
|---|---|---|---|---|---|---|
| Age-weeks | | | | | | |
| 14 | 15 | 16 | 17 | 18 | 20 | 22 |

0.5 cm Implants - treated at 20 weeks of age

| | | | |
|---|---|---|---|
| 20 | 1 | 5.89 | 0.87 |
| 24 | 1 | 2.54 | 1.17 |
| 26 | 1 | 11.3 | 0.15 |
| Mean | | 6.58 | 0.73 |
| 21 | 2 | 4.1 | 1.22 |
| 46 | 2 | 1.7 | 0.8 |
| 47 | 2 | 2.68 | 2 |
| Mean | | 2.83 | 1.34 |
| 29 | 3 | 3.84 | 1.39 |
| 44 | 3 | 4.59 | 1.89 |
| 45 | 3 | 4.2 | 0.8 |
| Mean | | 4.21 | 1.36 |

TABLE 2F

| | |
|---|---|
| 0.25 cm | Serum Testosterone - ng/ml |
| Implants | Treated at 20 weeks of age |

| | | | |
|---|---|---|---|
| 25 | 1 | 4.9 | 1.35 |
| 28 | 1 | 8.06 | 1.44 |
| 43 | 1 | 3.57 | 1.33 |
| Mean | | 5.51 | 1.37 |
| 22 | 2 | 5.07 | 2.57 |
| 23 | 2 | 7.92 | 1.44 |
| 30 | 2 | 4.41 | 1.57 |
| Mean | | 5.80 | 1.86 |
| 19 | 3 | 5.69 | 3.76 |
| 27 | 3 | 1.22 | 1.74 |
| 48 | 3 | 2.79 | 3.09 |
| Mean | | 3.23 | 2.86 |

### Conclusions: Example 1

It was concluded from this trial that treatment 3's formulation showed the most promise, particularly when only 25% of the 1 cm dose was used for treating pigs for 2 weeks between 20 and 22 weeks of age, with an implant of 0.25 cm.

A new experiment was designed to assess increasing the dosage of deslorelin while concentrating on the length of 0.25 cm by using a plurality of implants.

The vaccine formulations of example 1 were modified as described in Table 3 below.

### EXAMPLE 2

### Studies on the Release of Deslorelin Acetate from Slow Release Implants in Pigs

### Study Number: DSL-P-04-001

### Study Location: Pig Research and Training Centre (PRTC)

Victorian Institute of Animal Science
Department of Primary Industries
600 Sneydes Road
Werribee Victoria Australia 3030

### Implants:

Three different implants were evaluated.

Implant number 3 consisted of deslorelin acetate at 36% with no additives and an external diameter of 1.5 mm.
Implant number 4 consisted of deslorelin acetate at 54% with no additives and an internal diameter of 1.5 mm.
Implant number 5 consisted of deslorelin acetate at 36% with no additives and an external diameter of 2.0 mm.

### Size of Implants and Duration of Treatment:

Groups of pigs were treated with different implant lengths but all treatment added up to 1 cm of total implant. Pigs were treated for 2 or 4 weeks.

Pigs were treated with implants measuring 1 cm cut into lengths of 1 cm, 2×0.5 cm and 4×0.25 cm. Pigs were treated at 18 or 20 weeks of age. All pigs were sacrificed at 22 weeks of age. Thus pigs were treated for 2 or 4 weeks.

### Measurements:

The primary measure was the level of boar taint substances—androstenone and skatole in body fat. Peripheral blood samples were collected at various times after treatment for the measurement of serum testosterone. Daily feed intake, weekly weight gain and testicle size at slaughter was also measured.

The levels of androstenone and skatole in body fat of pigs were measured by VIAS Endocrinology HPLC Service VIAS SOP Number 840 (which has a minimum detection level for androstenone of 0.2 μg/g of fat, and for skatole of 0.3 μg/g of fat).

Serum testosterone measurements were performed by Fiona Armour, University of Melbourne Veterinary Clinic and Hospital using the Coat-A-Count® Total Testosterone radioimmunoassay supplied by Diagnostic Products Corporation, Los Angeles, Calif., United States of America (which has a minimum detection level of 0.2 ng/mL of serum).

TABLE 3
Treatment Groups

| | | Age at Implantation | | | | | |
|---|---|---|---|---|---|---|---|
| | | 18^{1,3} | | | 20^{2,3} | | |
| | | Weeks | | | Weeks | | |
| | | Implant Length | | | | | |
| Treatment groups | | | | 1 × | | | 1 × |
| Implant | | 4 × | 2 × | 1.0 | 4 × | 2 × | 1.0 |
| No | | 0.25 cm | 0.5 cm | cm | 0.25 cm | 0.5 cm | cm |
| 3 | 1.5 mm | 3 | 3 | 3 | 3 | 3 | 3 |
| | diameter, | | | | | | |
| | 36% | | | | | | |
| | deslorelin | | | | | | |
| | acetate | | | | | | |
| 4 | 1.5 mm | 3 | 3 | 3 | 3 | 3 | 3 |
| | diameter, | | | | | | |
| | 54% | | | | | | |
| | deslorelin | | | | | | |
| | acetate | | | | | | |
| 5 | 2.0 mm | 3 | 3 | 3 | 3 | 3 | 3 |
| | diameter, | | | | | | |
| | 36% | | | | | | |
| | deslorelin | | | | | | |
| | acetate | | | | | | |

Notes:
¹Pigs' blood sampled at 18, 20, 21 and 22 weeks of age.
²Pigs' blood sampled at 20, 21 and 22 weeks of age.
³Boar taint substances measured at 22 weeks of age.

### Animals and Groups:

Animals were sourced from PRTC and consisted of male large white/landrace pigs.

Pigs were housed in individual pens in the Experimental Grower Facility—PRTC. Identification—pigs were identified by the ear notches (placed at farrowing) and by ear tags.

### Results

The influence of deslorelin acetate on the elimination of boar taint substances in the fat of pigs and on testosterone levels in the serum of pigs are illustrated in Tables 4A to 4E.

TABLE 4A

| | | | | Treatment | | | | | | | | |
|---|---|---|---|---|---|---|---|---|---|---|---|---|
| | | | | 0.25 cm × 4 | | | 0.5 cm × 2 | | | 1.0 cm × 1 | | |
| Duration: 2 weeks | | | | Serum | | | Serum | | | Serum | | |
| | Pig No. | | | Testosterone | | | Testosterone | | | Testosterone | | |
| Implant | Implant Size | | | Weeks | | | Weeks | | | Weeks | | |
| No | 0.25 cm | 0.5 cm | 1.0 cm | 0 | 1 | 2 | 0 | 1 | 2 | 0 | 1 | 2 |
| 3 | 12 | 48 | 28 | 1.72 | 0.87 | 0.03 | 2.18 | 7.51 | 0.45 | 2.45 | 3.49 | 3.19 |
| | 16 | 49 | 32 | 4 | 0.79 | 0.66 | 1.8 | 3.51 | 0.31 | 3.67 | 3.67 | 1.46 |
| | 23 | 56 | 40 | 1.7 | 0.45 | 0.1 | 2.58 | 1.3 | 0.11 | 1.85 | 2.6 | 4.93 |
| 4 | 1 | 7 | 42 | 2.5 | 0.7 | 0.3 | 2.2 | 4.1 | 0.3 | 2.7 | 3.3 | 3.2 |
| | 5 | 9 | 47 | | | | | | | | | |
| | 8 | 11 | 50 | 3.67 | 0.34 | 0.08 | 2.93 | 1.5 | 0.82 | 1.73 | 1.55 | 2.63 |
| 5 | 3 | 6 | 33 | 1.91 | 0.43 | 0.12 | 1.16 | 2.66 | 2.39 | 2.89 | 2.03 | 1.67 |
| | 20 | 10 | 36 | 3.5 | 0.74 | 0.12 | 4.89 | 0.46 | 0.22 | 2.22 | | |
| | 22 | 14 | 38 | 3.0 | 0.5 | 0.1 | 3.0 | 1.5 | 1.1 | 2.3 | 1.8 | 2.2 |
| | | | | 6.81 | 2.08 | 0.21 | 1.89 | 1.34 | 0.74 | 0.95 | 0.83 | 0.92 |
| | | | | 2.46 | 2.58 | 3.72 | 4.11 | 1.55 | 0.14 | 2.99 | 8.26 | 3.9 |
| | | | | 3.33 | 3.56 | 3.47 | 4.31 | 0.86 | 2.36 | 3.41 | 3.03 | 2.75 |
| | | | | 4.2 | 2.7 | 2.5 | 3.4 | 1.3 | 1.1 | 2.5 | 4.0 | 2.5 |

TABLE 4B

| | | | | Treatment | | | | | | | | | | | |
|---|---|---|---|---|---|---|---|---|---|---|---|---|---|---|---|
| Duration: 4 weeks | | | | 0.25 cm × 4 | | | | 0.5 cm × 2 | | | | 1.0 cm × 1 | | | |
| | Pig No. | | | Serum Testosterone | | | | Serum Testosterone | | | | Serum Testosterone | | | |
| Implant | Implant Size | | | Weeks | | | | Weeks | | | | Weeks | | | |
| No | 0.25 cm | 0.5 cm | 1.0 cm | 0 | 2 | 3 | 4 | 0 | 2 | 3 | 4 | 0 | 2 | 3 | 4 |
| 3 | 13 | 69 | 2 | 4.87 | 0.7 | 0.7 | 0.44 | 2.51 | 2.8 | 2.47 | 3.43 | 0.15 | 0.64 | 0.36 | 4.55 |
| | 44 | 73 | 30 | 2.89 | 0.48 | 0.01 | 0.06 | 2.34 | 0.57 | 0.13 | 0.17 | 2.47 | 1.36 | 1.6 | 2.62 |
| | 68 | 75 | 67 | 2.05 | 0.16 | 0.09 | 0.09 | 1.87 | 2.15 | 1.66 | 5.01 | 4.36 | 1.71 | 0.62 | 0.99 |
| 4 | 34 | 17 | 25 | 3.3 | 0.4 | 0.3 | 0.2 | 2.2 | 1.8 | 1.4 | 2.9 | 2.3 | 1.2 | 0.9 | 2.7 |
| | 52 | 26 | 27 | | | | | | | | | | | | |
| | 55 | 74 | 72 | 2.38 | 0.05 | 0.31 | 0.08 | 4.33 | 0.07 | 0.05 | 0.08 | 2.21 | 0.74 | 0.77 | 0.24 |
| 5 | 29 | 19 | 18 | 0.8 | 0.04 | 0 | 0 | 5.93 | 0.09 | 0.14 | 0.33 | 4.51 | 2.85 | 1.09 | 1.5 |
| | 31 | 24 | 51 | 1.88 | 0.1 | 0.05 | 0.03 | 2.25 | 0.33 | 0.24 | 0.3 | 6.12 | 3.24 | 2.83 | 7.82 |
| | 53 | 70 | 71 | 1.7 | 0.1 | 0.1 | 0.0 | 4.2 | 0.2 | 0.1 | 0.2 | 4.3 | 2.3 | 1.6 | 3.2 |
| | | | | 2.41 | 2.41 | 4.03 | 4.45 | 7.35 | 3.18 | 0.79 | 9.2 | 2.16 | 0.08 | 0.04 | 0.03 |
| | | | | 1.41 | 1.34 | 1.28 | 3.19 | 3.69 | 3.47 | 2.64 | 6.64 | 1.35 | 1.92 | 1.83 | 2.93 |
| | | | | 4.31 | 5.73 | 3.73 | 3.68 | 3.5 | 3.26 | 2.47 | 6.94 | 1.98 | 1.14 | 2.15 | 4.39 |
| | | | | 2.7 | 3.2 | 3.0 | 3.8 | 4.8 | 3.3 | 2.0 | 7.6 | 1.8 | 1.0 | 1.3 | 2.5 |

TABLE 4C

| Duration: 2 weeks | | | | | | | | | | | | |
|---|---|---|---|---|---|---|---|---|---|---|---|---|
| | Pig No. | | | Treatment | | | | | | | | |
| Implant | Implant Size | | | 0.25 cm × 4 | | | 0.5 cm × 2 | | | 1.0 cm × 1 | | |
| No | 0.25 cm | 0.5 cm | 1.0 cm | Aldos | Skatole | Test-Fin | Aldos | Skatole | Test-Fin | Aldos | Skatole | Test-Fin |
| 3 | 12 | 48 | 28 | 0 | 0.1 | 0.03 | 0.5 | 0.5 | 0.45 | 1.4 | 0.24 | 3.19 |
| | 16 | 49 | 32 | 0.1 | 0.09 | 0.66 | 0.2 | 0.31 | 0.31 | 0.7 | 0.16 | 1.46 |
| | 23 | 56 | 40 | 2.1 | 0.5 | 0.1 | 0.4 | 0.11 | 0.11 | 1.1 | 0.43 | 4.93 |
| | | | | 0.73 | 0.23 | 0.3 | 0.4 | 0.3 | 0.3 | 1.1 | 0.3 | 3.2 |
| 4 | 1 | 7 | 42 | 0 | 0.06 | 0.08 | 0.3 | 0.17 | 0.82 | 1.2 | 0.09 | 2.63 |
| | 5 | 9 | 47 | 0 | 0.14 | 0.12 | 0.6 | 0.5 | 2.39 | 2 | 0.2 | 1.67 |
| | 8 | 11 | 50 | 0 | 0.08 | 0.12 | 0.3 | 0.18 | 0.22 | | | NS |
| | | | | 0.00 | 0.09 | 0.1 | 0.40 | 0.28 | 1.1 | 1.60 | 0.15 | 2.2 |
| 5 | 3 | 6 | 33 | 1.1 | 0.5 | 0.21 | 0 | 0.14 | 0.74 | 0.7 | 0.2 | 0.92 |
| | 20 | 10 | 36 | 1.4 | 0.16 | 3.72 | 0.3 | 0.12 | 0.14 | 2.5 | 0.12 | 3.9 |
| | 22 | 14 | 38 | 0.8 | 0.11 | 3.47 | 1 | 0.13 | 2.36 | 0.3 | 0.19 | 2.75 |
| | | | | 1.10 | 0.26 | 2.5 | 0.43 | 0.13 | 1.1 | 1.17 | 0.17 | 2.5 |

TABLE 4D

| Duration: 4 weeks | | | | | | | | | | | | |
|---|---|---|---|---|---|---|---|---|---|---|---|---|
| | Pig No. | | | Treatment | | | | | | | | |
| Implant | Implant Size | | | 0.25 cm × 4 | | | 0.5 cm × 2 | | | 1.0 cm × 1 | | |
| No | 0.25 cm | 0.5 cm | 1.0 cm | Aldos | Skatole | Test-Fin | Aldos | Skatole | Test-Fin | Aldos | Skatole | Test-Fin |
| 3 | 13 | 69 | 2 | 0 | 0.11 | 0.44 | 2.5 | 0.5 | 3.43 | 0.3 | 0.16 | 4.55 |
| | 44 | 73 | 30 | NS | NS | 0.06 | 0 | 0.09 | 0.17 | 0 | 0.15 | 2.62 |
| | 68 | 75 | 67 | 0 | 0.14 | 0.09 | 0.3 | 0.46 | 5.01 | 0.5 | 0.07 | 0.99 |
| | | | | 0.00 | 0.13 | 0.2 | 0.93 | 0.35 | 2.9 | 0.27 | 0.13 | 2.7 |
| 4 | 34 | 17 | 25 | 0 | 0.03 | 0.08 | 0 | 0.06 | 0.08 | 0.4 | 0.08 | 0.25 |
| | 52 | 26 | 27 | 0 | 0.06 | 0 | 0 | 0.31 | 0.33 | 1.5 | 0.04 | 1.5 |
| | 55 | 74 | 72 | 0 | 0.13 | 0.03 | 0 | 0.05 | 0.3 | 2.2 | .5 | 7.82 |
| | | | | 0.00 | 0.07 | 0.04 | 0.00 | 0.29 | 0.2 | 1.37 | 0.21 | 3.2 |
| 5 | 29 | 19 | 18 | 1.5 | 0.15 | 4.45 | 0.8 | 0.5 | 9.2 | 1.3 | 0.17 | 0.3 |
| | 31 | 24 | 51 | 0.6 | 0.34 | 3.19 | 2.5 | 0.5 | 6.64 | 0.6 | 0.23 | 2.93 |
| | 53 | 70 | 71 | 0.8 | 0.11 | 3.68 | 1.5 | 0.5 | 6.94 | 1 | 0.25 | 4.39 |
| | | | | 1.05 | 0.25 | 3.8 | 1.60 | 0.50 | 7.6 | 0.97 | 0.22 | 2.5 |

TABLE 4E
SUMMARY OF DSL-P-04-001
Use of deslorelin acetate to control boar taint

| | Testosterone (ng/mL) | | | | | | Skatole |
|---|---|---|---|---|---|---|---|
| | Week | Week | Week | Week | Week | Androstenone | ug/gram |
| Treatment | 0 | 1 | 2 | 3 | 4 | ug/gram fat | fat |
| Duration | | | | | | | |
| 2 weeks | | | | | | | |
| 0.25 cm × | 3.0 | 0.5 | 0.1 | | | 0.0 | 0.09 |
| 4 | | | | | | | |
| 1.5 mm | | | | | | | |
| (54%) | | | | | | | |
| Duration | | | | | | | |
| 4 weeks | | | | | | | |
| 0.25 cm × | 3.3 | | 0.4 | 0.3 | 0.2 | 0.0 | 0.13 |
| 4 | | | | | | | |
| 1.55 mm | | | | | | | |
| (36%) | | | | | | | |
| 0.25 cm × | 1.7 | | 0.1 | 0.1 | 0 | 0.0 | 0.07 |
| 4 | | | | | | | |
| 1.5 mm | | | | | | | |
| (54%) | | | | | | | |

The results achieved with the treatments described in the summary Table 4E for DSL-P-04-001 have not been observed previously using any form of treatment including that of castration. No evidence of testosterone reaching levels of 0-0.1 ng/mL in a 2 week period have been reported and similarly no reports of androstenone reaching levels of “0” in 2 to 4 weeks are present in the published literature. The levels of skatole achieved for all three preferred treatments reported in the summary table are significantly and consistently less than the desired <0.20 μg/gram fat and show that the reduction in testosterone and androstenone to near zero also results in significantly lowered skatole levels reflecting a direct effect of the treatments used.

### EXAMPLE 3

### Studies on the Release of Deslorelin Acetate from Slow Release Implants in Pigs

### Study Number: DSL-P-04-002

### Study Location: Pig Research and Training Centre (PRTC)

Victoria Institute of Animal Science
Department of Primary Industries
600 Sneydes Road
Werribee Victoria Australia 3030

### Implants:

The implants evaluated was as follows:

Implant formulation 2 consisted of deslorelin acetate at 54% with an internal diameter of 1.4 mm and an external diameter of 1.6 mm.

### Size of Implants and Duration of Treatment:

Three groups of pigs were treated as follows:
Group 1—10 male pigs—no treatment (control)
Group 2—5 male pigs—9.6 mg deslorelin—treated 2 weeks
Group 3—5 male pigs—9.6 mg deslorelin—treated 4 weeks

Pigs were treated with implants measuring 1 cm cut into lengths of 0.25 cm (ie; 4×0.25 cm).

Group 2 pigs were treated for 14 days (2 weeks) and
Group 3 pigs were treated for 28 days (4 weeks).

Pigs in Group 1 were 18-21 weeks of age at day 0, pigs in Group 2 were 21-22 weeks of age at day 0 and pigs in Group 3 were 18-22 weeks of age at day 0. In this experiment it was attempted to have all pigs finishing treatment in the same weight range of 120-125 kg.

### Measurements:

The primary measure was the level of boar taint substances—androstenone and skatole—in body fat. Peripheral blood samples were collected at various times after treatment for the measurement of serum testosterone. Daily food intake, weekly weight gain, average daily gain (ADG), feed conversion ratio (FCR), P2 depth and change in P2 over time were also measured.

The levels of androstenone and skatole in body fat of pigs were measured by VIAS Endocrinology HPLC Service VIAS SOP Number 840 (which has a minimum detection level for androstenone of 0.2 μg/g of fat, and for skatole of 0.3 μg/g of fat) and testosterone by VIAS Endocrinology Service using the Immulite® Total Testosterone Assay (Diagnostic Products Corporation, Los Angeles, Calif., United States of America) (which has a minimum detection level of 0.15 ng·mL of serum).

### Animals and Groups:

Animals were sourced from PRTC and consisted of male large white/landrace pigs.

Pigs were housed in individual pens in the Experimental Grower Facility—PRTC. Pigs were identified by ear notches (placed at farrowing) and by ear tags.

### Result:

The influence of desorelin acetate on the elimination of boar taint substances in the fat of pigs and on testosterone, androstenone and skatole levels in the serum of pigs are illustrated in Tables 5A-5D, along with food conversion ratios (FCR), average daily gain (ADG) and fat depth (P2).

TABLE 5A
Group 1: Control pigs - no treatment - entire male animals

| | | | | | | Serum Testosterone | | | | |
|---|---|---|---|---|---|---|---|---|---|---|
| | | | | | | (ng/mL) | | | | |
| Pig | FCR at | FCR at | ADG at | ADG at | Change in | Days - Postimplantation | | | Andros | Skatole |
| No | 7 days | 14 days | 7 days | 14 days | P2 (mm) | 0 | 7 | 17 | ug/g | ug/g |
| 5 | 2.38 | 2.92 | 1.1 | 1.0 | 2 | 4.64 | 3.03 | 7.30 | 2.4 | 0.34 |
| 7 | 3.05 | 3.22 | 0.9 | 0.9 | 0.5 | 6.29 | 3.92 | 4.27 | 2.4 | 0.4 |
| 8 | 2.31 | 2.57 | 1.3 | 1.2 | 0 | 15.86 | 6.40 | 4.59 | 2.4 | 0.6 |
| 9 | 2.78 | 2.91 | 0.9 | 1.0 | 3 | 4.67 | 3.23 | 2.48 | NS | NS |
| 11 | 2.35 | 2.64 | 1.1 | 1.1 | 1 | 12.00 | 15.14 | 6.23 | NS | NS |
| 12 | 2.34 | 2.59 | 1.2 | 1.2 | 2 | 7.85 | 7.36 | 3.37 | NS | NS |
| 13 | 2.38 | 2.47 | 1.2 | 1.2 | 0.5 | 4.47 | 6.03 | 4.93 | NS | NS |
| 20 | 4.87 | 3.54 | 0.7 | 1.1 | 2.5 | 5.94 | 5.68 | 4.96 | NS | NS |
| 28 | 2.33 | 2.63 | 1.2 | 1.1 | 2 | 6.32 | 4.79 | 4.87 | 2.4 | 0.17 |
| 36 | 5.33 | 4.08 | 0.6 | 0.7 | 1.5 | 7.21 | 6.78 | 15.86 | 2.4 | 0.27 |
| Mean | 3.01 | 2.96 | 1.03 | 1.04 | 1.5 | 7.53 | 6.24 | 5.89 | 2.4 | 0.4 |

NS = Not Sampled

TABLE 5B
Group 2: 4 × 0.25 cm implants (54% deslorelin acetate) - two weeks duration

| | | | | | | Serum Testosterone | | | | |
|---|---|---|---|---|---|---|---|---|---|---|
| | | | | | | (ng/mL) | | | | |
| Pig | FCR at | FCR at | ADG at | ADG at | Change in | Days - Postimplantation | | | Andros | Skatole |
| No | 7 days | 14 days | 7 days | 14 days | P2 (mm) | 0 | 7 | 14 | ug/g | ug/g |
| 16 | 3.18 | 3.18 | 1.00 | 1.13 | 1.5 | 4.79 | 0.00 | 0.32 | 0 | 0.03 |
| 24 | 3.47 | 3.34 | 0.83 | 0.91 | 1.5 | 3.95 | 0.69 | 0.00 | 0 | 0.07 |
| 26 | 4.31 | 3.77 | 0.80 | 0.97 | 4 | 4.27 | 0.32 | 0.29 | 0 | 0.08 |
| 38 | 2.33 | 2.63 | 1.23 | 1.10 | −0.5 | 8.34 | 0.40 | 0.00 | 0.26 | 0.15 |
| 43 | 3.83 | 3.62 | 0.63 | 0.77 | 1.5 | 15.66 | 0.26 | 0.00 | 0.24 | 0.03 |
| Mean | 3.43 | 3.31 | 0.90 | 0.98 | 1.6 | 7.40 | 0.33 | 0.12 | 0.10 | 0.07 |

TABLE 5C
Group 1: Control pigs - no treatment - entire male animals

| | | | | | | Serum Testosterone | | | | | |
|---|---|---|---|---|---|---|---|---|---|---|---|
| | | | | | | (ng/mL) | | | | | |
| Pig | FCR at | FCR at | ADG at | ADG at | Change in | Days - Postimplantation | | | | Andros | Skatole |
| No | 14 days | 28 days | 14 days | 28 days | P2 (mm) | 0 | 7 | 17 | 28 | ug/g | ug/g |
| 5 | 2.92 | 2.82 | 1.0 | 1.1 | 6 | 4.64 | 3.03 | 7.30 | 8.91 | 2.4 | 0.34 |
| 7 | 3.22 | 2.77 | 0.9 | 1.1 | 2.5 | 6.29 | 3.92 | 4.27 | 10.15 | 2.4 | 0.4 |
| 8 | 2.57 | 2.44 | 1.2 | 1.3 | 4 | 15.86 | 6.40 | 4.59 | 5.36 | 2.4 | 0.6 |
| 9 | 2.91 | 2.99 | 1.0 | 1.1 | 6 | 4.67 | 3.23 | 2.48 | 2.83 | NS | NS |
| 11 | 2.64 | 2.67 | 1.1 | 1.0 | 2.5 | 12.00 | 15.14 | 6.23 | 8.25 | NS | NS |
| 12 | 2.59 | 2.87 | 1.2 | 1.1 | 3.5 | 7.85 | 7.36 | 3.37 | 5.68 | NS | NS |
| 13 | 2.47 | 2.63 | 1.2 | 1.1 | 3 | 4.47 | 6.03 | 4.93 | 8.62 | NS | NS |
| 20 | 3.54 | 3.28 | 1.1 | 1.2 | 7 | 5.94 | 5.68 | 4.96 | 11.19 | NS | NS |
| 28 | 2.63 | 2.65 | 1.1 | 1.1 | 5 | 6.32 | 4.79 | 4.87 | 8.42 | 2.4 | 0.17 |
| 36 | 4.08 | 3.54 | 0.7 | 0.9 | 2 | 7.21 | 6.78 | 15.86 | 4.70 | 2.4 | 0.27 |
| Mean | 2.96 | 2.87 | 1.04 | 1.08 | 4.2 | 7.53 | 6.24 | 5.89 | 7.41 | 2.4 | 0.4 |

NS = Not Sampled

TABLE 5D
Group 3: 4 × 0.25 cm implants (54% deslorelin acetate) - four weeks duration

| | | | | | | Serum Testosterone | | | | | |
|---|---|---|---|---|---|---|---|---|---|---|---|
| | | | | | | (ng/mL) | | | | | |
| Pig | FCR at | FCR at | ADG at | ADG at | Change in | Days - Postimplantation | | | | Andros | Skatole |
| No | 14 days | 28 days | 14 days | 28 days | P2 (mm) | 0 | 7 | 14 | 28 | ug/g | ug/g |
| 15 | 2.48 | 2.78 | 1.10 | 1.20 | 5.5 | 14.71 | 0.00 | 0.00 | 0.00 | 0 | 0.11 |
| 18 | 3.14 | 3.82 | 1.26 | 1.14 | 6.5 | 9.26 | 0.00 | 0.00 | 0.52 | 0 | 0.04 |
| 32 | 2.32 | 3.06 | 1.26 | 1.16 | 3.0 | 8.16 | 0.23 | 0.00 | 0.32 | 0 | 0.04 |
| Mean | 2.64 | 3.22 | 1.20 | 1.17 | 5.0 | 10.7 | 0.08 | 0.0 | 0.28 | 0.0 | 0.06 |

TABLE 5E
Average deslorelin acetate levels measured in implants recovered
from pigs 2 and 4 weeks after implantation

| | | |
|---|---|---|
| Starting Amount (mg) | Finishing Amount (mg) | % Released |

2 week release

| | | |
|---|---|---|
| 2.4 | 1.6 | 33.3 |

4 week release

| | | |
|---|---|---|
| 2.4 | 1.1 | 54.2 |

Selected implants that were removed from pigs in Group 2 and Group 3 revealed that over the 2 and 4 week treatment periods approximately 33% and 54% of the active deslorelin acetate was released from the implants (see Table 5E).

The results achieved with the treatments described in this example have not been observed previously using any other form of treatment, including that of castration. No evidence of serum testosterone reaching levels of 0-0.12 ng/mL in a two week period have been reported and similarly no reports of androstenone reaching levels of 0-0.1 μg/mL in 2 to 4 weeks are present in the published literature. The levels of skatole achieved are significantly and consistently below the desired <0.2 μg/g of fat and show that the reduction in testosterone and androstenone to near zero also results in significantly lowered skatole levels, reflecting a direct effect of the treatments used. The simultaneous reduction in the levels of all three of serum testosterone, androstenone and skatole has not been shown in the published literature.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and may be used interchangeably and should not be taken as excluding the presence of other elements or features.

1. A sustained release apparatus including at least one sustained release mini-implant or pellet;
the or each mini-implant or pellet including;
a sustained release support material; and
a pharmaceutical composition including
a Luteinising Hormone Releasing Hormone (LHRH) agonist and/or antagonist component
the size and/or number and/or payload of mini-implant(s) or pellet(s) providing, release of LHRH agonist and/or antagonist at, or above, a desired threshold level for treatment of a selected indication, the apparatus providing approximately zero order release of the LHRH agonist and/or antagonist.

2. A sustained release apparatus according to claim 1 including a plurality of mini-implants or pellets which in combination provide a blood level of LHRH agonist and/or antagonist at least equal to a predetermined threshold for an extended period.

3. A sustained release apparatus according to claim 2 wherein the extended period is approximately 1 to 52 weeks.

4. A sustained release apparatus according to claim 3 wherein the extended period is approximately 2 to 4 weeks.

5. A sustained release apparatus according to claim 1 wherein the total LHRH agonist and/or antagonist content is approximately 1 to 20 mg.

6. A sustained release apparatus according to claim 1 wherein the length of the or each mini-implant or pellet is approximately 0.05 to 1.5 cm.

7. A sustained release apparatus according to claim 1 wherein the internal diameter of the or each mini-implant or pellet is approximately 0.5 to 2.0 mm.

8. A sustained release apparatus according to claim 1 wherein the LHRH agonist and/or antagonist component is an active derivative or active fragment of LHRH.

9. A sustained release apparatus according to claim 1 wherein the LHRH agonist is selected from the group consisting of [D-Trp6] LHRH, Decapeptyl, Leuprolide, Zolandex, Buserelin, or Deslorelin (D-Trp⁶-Pro⁹-des-Gly¹⁰-LHRH ethylamide), and salts thereof.

10. A sustained release apparatus according to claim 1 wherein the LHRH antagonist is selected from the group consisting of Ganirelix, Abarelix, Cetrorelix acetate (Ac-D-NaI(2)(4CI), D-Pal(3)3, D-Cit6, D-Ala10) LHRH or Cetrorelix pamoate, and salts thereof.

11. A sustained release apparatus according to claim 1 wherein the pharmaceutical composition further includes a carrier selected from synthetic polymers, sugars, polysaccharides, amino acids, mineral salts, organic salts, proteins, resins, latexes, waxes and lipids.

12. A sustained release apparatus according to claim 11 wherein the pharmaceutical composition further includes a carrier selected from the group consisting of lactose, sucrose, sodium deoxycholic acid, mannitol, sodium chloride and dextran.

13. A sustained release apparatus according to claim 11 wherein the carrier is present in amounts of from approximately 1 to 30% by weight, based on the total weight of the pharmaceutically active composition.

14. A sustained release apparatus according to claim 1 wherein, in use in entire male animals, the blood level of LHRH agonist and/or antagonist is sufficient to significantly reduce skatole levels.

15. A sustained release apparatus according to claim 14 wherein, in use in entire male animals, the blood level of LHRH agonist and/or antagonist is sufficient to concomitantly significantly reduce testosterone levels.

16. A sustained release apparatus according to claim 15 wherein, in use in entire male animals, the blood level of LHRH agonist and/or antagonist is sufficient to concomitantly significantly reduce androstenone levels.

17. A sustained release apparatus according to claim 1 wherein the pharmaceutical composition further includes a secondary pharmaceutically active component selected from a water-insoluble pharmaceutical active, a water-soluble pharmaceutical active or mixtures thereof.

18. A sustained release apparatus according to claim 17 wherein the secondary pharmaceutically active component is a natural or synthetic growth hormone.

19. A sustained release apparatus according to claim 17 wherein the secondary pharmaceutically active component is present in amounts from approximately 8 to 50% by weight, based on the total weight of the pharmaceutical composition.

20. A sustained release apparatus according to claim 2 wherein the apparatus includes two or more mini-implants of similar or different sizes.

21. A sustained release apparatus according to claim 1 wherein each sustained release mini-implant or pellet is of the covered rod or matrix type.

22. A sustained release apparatus according to claim 21 wherein the sustained release support material is in the form of a covered rod structure or an open ended cylindrical rod.

23. A sustained release apparatus according to claim 1 wherein the sustained release support material is a silicone material.

24. A sustained release apparatus according to claim 1 wherein the sustained release support material is present in amounts of approximately 40 to 65% by weight, based on the total weight of the apparatus.

25. A sustained release apparatus including at least one sustained release mini-implant or pellet;
the or each mini-implant or pellet including;
a sustained release support material; and
a pharmaceutical composition including
a Luteinising Hormone Releasing Hormone (LHRH) agonist and/or antagonist component;
the size and/or number and/or payload of mini-implant(s) or pellet(s) providing, release of LHRH agonist and/or antagonist at, or above, a desired threshold level for regulation of characteristics associated with the onset of sexual maturation of an animal and/or seasonal breeding activity, the apparatus providing a blood level of LHRH agonist and/or antagonist sufficient to significantly reduce skatole levels.

26. A sustained release apparatus according to claim 25 wherein the blood level of LHRH agonist and/or antagonist is sufficient to concomitantly significantly reduce testosterone levels.

27. A sustained release apparatus according to claim 26 wherein the blood level of LHRH agonist and/or antagonist is sufficient to concomitantly significantly reduce androstenone levels.

28. A sustained release apparatus according to claim 25 wherein the total LHRH agonist and/or antagonist content is approximately 1 to 20 mg.

29. A sustained release apparatus according to claim 28 wherein the total LHRH agonist and/or antagonist content is approximately 2 to 6 mg.

30. A sustained release kit including
a plurality of sustained release mini-implants or pellets packaged for delivery in a single treatment;
each mini-implant or pellet including
a sustained release support material; and
a pharmaceutical composition including
a LHRH agonist and/or antagonist component;
the size and/or number and/or payload of mini-implants or pellets providing, in use, release of agonist and or antagonist at, or above, a desired threshold level for treatment of a selected indication, the plurality of sustained release mini-implants or pellets providing approximately zero order release of the agonist and/or antagonist.

31. A sustained release kit according to claim 30 including 3 to 12 mini-implants or pellets.

32. A sustained release kit according to claim 30 further including a sustained release delivery apparatus.

33. A sustained release apparatus according to claim 30 wherein the plurality of sustained release mini-implants or pellets is provided in a biodegradable sheath.

34. A method for the therapeutic or prophylactic treatment of an indication in an animal, including humans, requiring such treatment, which method includes administering to the animal a sustained release apparatus including one or more sustained release mini-implants or pellets;
the or each mini-implant or pellet including
a sustained release support material; and
a pharmaceutical composition including
an LHRH agonist and/or antagonist component;
the size and/or number and/or payload of mini-implants or pellets providing release of agonist and/or antagonist at, or above, a desired threshold level for treatment of a selected indication, the apparatus providing approximately zero order release of the agonist and/or antagonist.

35. A method according to claim 34 wherein the sustained release apparatus includes a plurality of mini-implants or pellets which in combination provide a blood level of LHRH agonist and/or antagonist at least equal to a predetermined threshold for an extended period.

36. A method according to claim 34 wherein the treatment provides a reduction in hormone production or production of hormone-associated compounds.

37. A method according to claim 34 wherein the sustained release apparatus is administered orally or by subcutaneous or intramuscular injection, intradermal injection, intraperitoneal injection, intraocular or in the ear, intranasal insertion or indwelling, intravaginal or intradwelling or intrarectal insertion or indwelling.

38. A method according to claim 34 wherein the animal to be treated is selected from the group consisting of sheep, cattle, goats, horses, camels, pigs, dogs, cats, ferrets, rabbits, marsupials, buffalos, yacks, primates, humans, birds, rodents, fish and reptiles.

39. A method according to claim 34 wherein the treatment provides regulation of reproduction and the control and treatment of the gonads and other reproduction organs.

40. A method according to claim 34 wherein the indication is selected from the group consisting of prostate cancer, testicular cancer, prostate enlargement, ovarian cancer and endometriosis.

41. A method according to claim 34 wherein the treatment provides regulation of characteristics associated with the onset of sexual maturation of an animal and/or seasonal breeding activity.

42. A method according to claim 41 which method includes orally administering pellets to cattle.

43. A method for regulating sexual reproduction in animals, including humans, which method includes administering to the animal to be treated, a sustained release apparatus including at least one sustained release mini-implant or pellet;
the or each mini-implant or pellet including;
a sustained release support material; and
a pharmaceutical composition including
an LHRH agonist and/or antagonist component;
the size and/or number and/or payload of mini-implants or pellets providing release of agonist at, or above, a desired threshold level for treatment of a selected indication, the apparatus providing approximately zero order release of the agonist and/or antagonist.

44. A method according to claim 43 wherein the sustained release apparatus includes a plurality of mini-implants or pellets which in combination provide a blood level of LHRH agonist and/or antagonist at least equal to a predetermined threshold for an extended period.

45. A method of inhibiting the growth of cells which are regulated, directly or indirectly by LHRH, which method includes administering to the animal to be treated, a sustained release apparatus including at least one sustained release mini-implant or pellet;
the or each mini-implant or pellet including;
a sustained release support material; and
a pharmaceutical composition including
a LHRH agonist and/or antagonist component;
the size and/or number and/or payload of mini-implants or pellets providing release of agonist and/or antagonist at, or above, a desired threshold level for treatment of a selected indication the apparatus providing approximately zero order release of the agonist and/or antagonist.

46. A method according to claim 45 wherein the sustained release apparatus includes a plurality of mini-implants or pellets which in combination provide a blood level of LHRH agonist and/or antagonist at least equal to a predetermined threshold for an extended period.

47. A method according to claim 45 wherein the cells are selected from testicular cells, breast cells, prostate cells, ovarian cells, or oncofoetal cells.

48. A method for improving carcass quality in animals, which method includes
administering to an entire animal to be treated at a prescribed time and for a preselected short duration, a sustained release apparatus including at least one sustained release mini-implant or pellet;
the or each mini-implant or pellet including;
a sustained release support material; and
a pharmaceutical composition including
a LHRH agonist and/or antagonist component;
the size and/or number and/or payload of mini-implants or pellets providing release of agonist and/or antagonist at, or above, a desired threshold level for treatment of a selected indication, the apparatus providing approximately zero order release of agonist and/or antagonist.

49. A method according to claim 48 wherein the sustained release apparatus includes a plurality of mini-implants or pellets which in combination provide a blood level of LHRH agonist and/or antagonist at least equal to a predetermined threshold for an extended period.

50. A method of regulating characteristics associated with the onset of sexual maturation of an animal and/or seasonal breeding activity, which method includes administering to the animal to be treated, a sustained release apparatus including at least one sustained release mini-implant or pellet;
the or each mini-implant or pellet including;
a sustained release support material; and
a pharmaceutical composition including
an LHRH agonist and/or antagonist component;
the size and/or number and/or payload of mini-implant(s) or pellet(s) providing, release of LHRH agonist and/or antagonist sufficient to produce a blood level thereof which significantly reduces skatole levels.

51. A method according to claim 50 wherein the blood level of LHRH agonist and/or antagonist is sufficient to concomitantly significantly reduce testosterone levels.

52. A method according to claim 51 wherein the blood level of LHRH agonist and/or antagonist is sufficient to concomitantly significantly reduce androstenone levels.

53. A method according to claim 50 wherein the apparatus provides approximately zero order release of the LHRH agonist and/or antagonist.

54. A method of reducing or eliminating boar taint which method includes administering to an entire male pig at a prescribed time and for a preselected short duration, a sustained release apparatus including at least one sustained release mini-implant or pellet;
the or each mini-implant or pellet including;
a sustained release support material; and
a pharmaceutical composition including
a LHRH agonist and/or antagonist component;
the size and/or number and/or payload of mini-implants or pellets providing release of LHRH agonist and/or antagonist sufficient to produce a blood level thereof which significantly reduces skatole levels

55. A method according to claim 54 wherein the apparatus provides approximately zero order release of the LHRH agonist and/or antagonist.

56. A method according to claim 55 wherein the blood level of LHRH agonist and/or antagonist is sufficient to concomitantly significantly reduce testosterone levels.

57. A method according to claim 56 wherein the blood level of LHRH agonist and/or antagonist is sufficient to concomitantly significantly reduce androstenone levels.

58. A method according to claim 57 wherein the blood level of LHRH agonist and/or antagonist is sufficient to reduce skatole levels to below 0.2 μg/g of fat.

59. A method according to claim 58 wherein the blood level of LHRH agonist and/or antagonist is sufficient to concomitantly reduce testosterone levels to below 1.0 ng/mL of serum.

60. A method according to claim 59 wherein the blood level of LHRH agonist and/or antagonist is sufficient to concomitantly reduce androstenone levels to below 0.5 μg/g of fat.

61. A method according to claim 60 wherein the blood level of LHRH agonist and/or antagonist is sufficient to reduce testosterone levels to below 0.2 ng/mL of serum.

62. A method according to claim 61 wherein the blood level of LHRH agonist and/or antagonist is sufficient to reduce androstenone levels to below a 0.2 μg/g of fat.

63. A pig carcass, or part thereof, substantially free of boar taint produced by a method according to claim 54.
